(19) United States
(12) Patent Application Publication
(10) Pub. No.: US 2010/0055516 A1
(43) Pub. Date: Mar. 4, 2010

(54) NICKEL SUBSTRATES

(76) Inventors: Geoffrey Mason, Leicestershire (GB); Bryan Buffham, Derby (GB); Klaus Hellgardt, London (GB); Paul Russell, Cleveland (GB); David Richardson, Leicestershire (GB)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/312,097

(22) PCT Filed: Oct. 25, 2007
(86) PCT No.: PCT/GB2007/004074

§ 371 Date: Nov. 2, 2009

(30) Foreign Application Priority Data
Oct. 27, 2006 (GB) 0621386.2

(51) Int. Cl. C23C 8/24; B01J 27/24; C07C 51/36; C08B 37/00; H01M 8/04; H01M 8/02; C25D 7/00

(52) U.S. Cl. 429/17; 148/238; 148/317; 502/200; 585/752; 536/124; 554/141; 429/34; 205/150

(57) ABSTRACT

A method of providing a porous surface on a nickel substrate comprising treating the substrate with a flowing stream of gas comprising ammonia or hydrazine at a temperature of at least 4000 C, the resultant porous surface comprising pores which are substantially all interconnected and have access to the surface.

This invention relates to the provision of new and useful surfaces on nickel substrates, such as but not limited to wires or spheres, as well as to usages of such metal substrates.

It is known to treat the nickel surfaces with a gas containing at least some ammonia at elevated temperatures. Indeed, a number of industrial processes utilize processes or reactions which comprise these bare elements.

For example, in “Nitridation in NH₃—H₂O mixtures”, Grabke et al, Materials and Corrosion 2003, 54, No. 11, there is described the exposure of nickel sheets at 500° C. to a flowing 1 bar gaseous mixture comprising 70% ammonia and 30% water for up to 200 hours. It is said that nickel nitride was observed, which is very unstable and immediately decomposes to nickel and nitrogen. Additionally, the nitrogen evolved at high pressure was said to cause pore formation, with internal nitridation and pore growth said to lead to internal stresses and surface cracks. The presence of a relatively high level of water in the treatment gas stream, together with the discussion in the paper of oxidation mechanisms, suggests oxidation has occurred at the resultant surface.

GB-A-1183642 (International Nickel Limited) describes the production of porous metal products (e.g. nickel) by impregnating a natural cellulosic fibrous material with a solution of a thermally decomposing metal salt. The solution enters the fibres, is dried and then treated to destroy the cellulosic material and decompose the salt to metal, resulting in a mass which is sintered into a coherent body. A preferred sintering atmosphere is cracked ammonia; conveniently sintering is carried out at a temperature of at least 900° C., and in some embodiments at least 1000° C. The resultant products are typically flexible sheets or meshes.

“Permeation of nitrogen in solid nickel and deformation phenomena accompanying internal nitridation,” Kodentsov et al, Acta Mater Vol 47, No. 11, pp 3169-3180, 1999, describes the internal nitridation of nickel (99.98%) alloys at 600-700° C. in a flowing 1 bar mixture of 15 vol % ammonia and 85 vol % hydrogen gas, the alloys containing 5 at. % chromium or 1.5 at. % titanium. The abstract of the paper further describes a large dislocation density generated in the nickel matrix upon the internal precipitation of semi-coherent nitride particles.

In more detail, the paper refers to the diffusion bonding of dense Si₃N₄ ceramic to nickel at elevated temperatures, and the build up of nitrogen, which is formed as a side-product of the interfacial reaction, building up a pressure (fugacity) at the metal/ceramic contact surface. It is postulated that the nitrogen gas has to escape from the reaction interface either along the contact surface through channels of connected pores, or by interstitial diffusion through the nickel-based solid solution.

In the discussion of the results of this experiment, references to the chromium or titanium nitride predominate, since for example in the context of the 5 at. % Cr alloy, it is said that “ . . . CrN is the only thermodynamically stable nitride, which may form in the reaction between nitrogen and the Ni 5 at. % Cr solid solution”. Formation of CrN is also said to be accompanied by volume change in the substrate. Discussion of nitrogen in relation to the nickel substrate focuses on discussing the solubility of nitrogen in nickel; typical N₂ fugacities of 27500 bar are suggested.

Changes in the form of the substrate are also described. For example in the context of Ni 5 at. % Cr alloy nitrided in flowing ammonia at 700° C., surface nodules (protuberances) consisting of virtually pure nickel within the grains are described, which are said to form indiscriminately over the grain surface during nitriding of the Ni—Cr solid solution. Samples of the nitrided alloy are also said to be covered with a thick layer of pure nickel which was probably formed by coalescence of nickel nodules “extruded” from the interior of the nitrided sample to the gas/metal contact surface. Overall though, the paper is silent on the formation, possible existence or contribution of any nickel nitride materials.

In a first aspect of the invention, there is provided a method of providing a porous surface on a nickel substrate comprising treating the substrate with a flowing stream of gas comprising ammonia or hydrazine at a temperature of at least 400° C.

Also provided according to the invention is a nickel substrate prepared according to the method of the invention.

In a further aspect, nickel substrates produced according to the invention are characterised by one or more (i.e. combinations) of the following features:
(a) they have a surface porous layer up to about 30 microns thick, sometimes only 10 or 20 microns thick;
(b) the surface has cracks along the grain boundaries;
(c) the pores have a surface pore diameter of less than 10 microns; preferably less than 5 microns, more preferably less than about 1 micron;
(d) substantially all (e.g. 90% or more, preferably 95% or more preferably 99% or more, preferably 99.9% or more) of the pores have access to the surface;
(e) that has been produced by a gas stream that contains less than 30% water;
(f) that has been produced in a gas stream which does not contain an oxidising or sulphiding agent;
(g) that the pores form an interconnected network;
(h) that the resultant surface resembles an open sponge.

By “nickel substrate” in the context of the invention is meant a nickel item having a surface on which the porous surface may be produced. The nickel substrate may be made of “pure” nickel (e.g. greater than 98.5% purity, more preferably greater than 99% purity, more preferably greater than 99.5% pure, even more preferably greater than 99.9% pure), or it may in some embodiments be a nickel alloy, or in other embodiments a nickel based material (e.g. one comprising at least 70% nickel). The term “nickel substrate” includes items which are nickel or nickel based throughout, but also includes items made of other materials which have a nickel surface coating.

Conveniently, the temperature of the gas stream is in the range 400° C. to 1100° C., more preferably at least 450° C., preferably in the range 550° C. to 650° C., and in a preferred embodiment around 600° C.

In relation to the flow rate of the gas stream used in the process of the invention, preferred flow rates will depend on other variables of the process, such as temperature, pressure and gas composition.

In a highly preferred aspect of the invention, the gas stream is free of oxidising agents and sulphiding agents. These may interact with the nickel on the surface being treated to form by-products such as nickel oxide or nickel sulphide which can be detrimental to use of the resultant substrates as catalysts. It may also optionally comprise an inert gas such as argon. It has also found to be important to the process of the invention that the gas stream flows during the treatment regime.

Conveniently, the treatment time of the nickel substrate with the gas flow is in the range 10 to 1000 hours, preferably 50 to 200 hours. However, the treatment time will be dependent on other aspects such as the concentration of ammonia or hydrazine in the gas stream and treatment temperature, and could in certain circumstances be up to about 1000 hours or more.

Conveniently, the gas pressure in the range 1 to 5 bar, preferably 1 bar, although higher pressures (e.g. up to about 1000 bar) can be used without detriment.

Preferably, the gas stream comprises at least 30% ammonia or hydrazine, more preferably at least 50%, at least 60%, at least 75%, or at least 90% or 95% ammonia or hydrazine. Most preferably, the gas stream is at least 99%, more preferably at least 99.5% ammonia or hydrazine. Such concentrations may be beneficial in the initial preparation of a nickel substrate according to the invention.

However, it is envisaged that when used in certain embodiments, for example in the operation of a reactor/fuel cell, the surface structure of the nickel substrate may be kept refreshed by substantially lower concentrations of ammonia or hydrazine. Concentrations of ammonia or hydrazine may be as low as 0.01%, conveniently more than 0.1% in the process gas and may be added continuously or periodically in order to “refresh” the catalyst or to provide the benefits outlined immediately above.

In a preferred embodiment, the “treatment” gas in the gas stream comprises ammonia.

Without wishing to be bound by theory, it is thought that the porous surface of nickel substrates, together with the other features of nickel substrates produced according to the invention such as grain cracks and pores of the size observed, are produced by the production in the process of the invention of unstable nickel nitride (Ni₃N). Nickel nitride is generated from nickel and ammonia, but is unstable above approximately 450° C. It is believed that with the relatively high level of ammonia in the gas stream, this causes a very high rate of decomposition of ammonia on the nickel surface, which in turn generates a huge fugacity which causes nitrogen to diffuse into the solid nickel surface. As a result, transient nickel nitride is generated. However, once inside the outer surface of the nickel substrate, it is away from the very high nitrogen fugacity at the surface of the nickel substrate which stabilizes it. The nickel nitride subsequently decomposes, releasing nitrogen gas at very high pressure generating a porous layer, the pores carrying a net outward flow.

The porous surface on the nickel substrate may have a number of beneficial advantages. Nickel is a commonly used industrial catalyst, and nickel produced according to the invention typically has a ten-fold increase in surface area associated with it. As such nickel substrates produced accordingly to the invention are beneficially used as catalysts, since the increase in surface area of the substrate results in an increase in catalytic activity. These may be used in industrial activities for which nickel catalysts are utilized; historically this has included processes such as the steam reforming of hydrocarbons, dry reforming of biogas, hydrogenation of sugars, the activation of fuel in high temperature fuel cell devices, and the catalytic hydrogenation of fatty acids in oils and fats.

A further preferred utility may be in Raney nickel catalysts, in which aluminium is leached out of a nickel aluminium alloy to produce a porous nickel powder, which is used for example as a hydrogenation catalyst. Raney nickel is a preferred industrial catalyst because of its stability and high catalytic activity at room temperature. It is typically used in the reduction of compounds that have multiple bonds such as alkynes, alkenes, nitrites, dienes, aromatics and carbonyls; Raney nickel additionally reduces heteroatom-heteroatom bonds such as nitro groups and nitrosamines. It has also found use in the reductive alkylation of amines, and in the amination of alcohols.

In a further embodiment, the beneficial catalytic activity may be obtained with supported nickel catalysts, as well as unsupported nickel catalysts. The method of the invention may also be used to regenerate existing nickel catalysts.

In yet a further embodiment, nickel produced according to the invention can be generated in the form of foams by processes and techniques known in the art and marketed e.g. by Inco, and may be combined e.g. with yttria-zirconia to increase the area of the metal-solid-gas interface in fuel cell membranes.

In more detail, in the context of high temperature fuel cells, one aim is to increase the three-phase boundary, gas/electrode/electrolyte, of the anode side. Currently nickel cermets are used for this purpose.

These are a mixture of electrolyte, such as yttria stabilized zirconia (YSZ) and nickel, to make it conducting. Nickel foam can be used to fill the space in the YSZ powder; if it is nickel foam produced according to the invention and the space filled with YSZ powder, the three-phase boundary may increase and therefore increase current density. Indeed, the use of ammonia instead of hydrogen in fuel cells increases current densities.

In addition, the invention can be used to increase the surface area of nickel powders in nicad battery plates and in nickel-hydride batteries.

In further utilities of the invention, the invention may be used to reduce the size of pores using electroless nickel plating of a pre-existing coarse porous substrate. The deposited thin nickel film may then be exposed to ammonia according to the invention to provide the required pore density. The resultant membranes may have sub-micron pores, and may be useful in micro-filtration.

In further envisaged utilities, the nickel substrates produced according to the invention may be used to provide a cost-efficient means of producing hydrogen fuel cells using renewable energy sources.

The nickel substrates may also be used as catalysts in aqueous processing systems for steam reforming, methanation and hydrogenation.

The invention may also be used to produce porous nickel foils, which are applicable to biomedical, sensor, magnetic and energy related materials. In particular, the porous structure may be biocompatible, and may deform into many different shapes. Its mechanical strength can also be enhanced, giving it significant advantages over conventional structures used to culture or grow cells.

Porous nickel substrates according to the invention with large internal surface areas can be used to make inter alia batteries, fuel cells, capacitors and sensors. They can also be used in photonic crystal and optical applications.

The invention can also be used to make porous shape memory articles, such as those made e.g. from nitinol.

The invention may also be utilized to make fuel cells to replace batteries in wireless applications, such as for example laptop computers and cellphones, as well as in oil refinery catalysts.

Nickel substrates made according to the invention may take any form including but not limited to spheres, wires, powder, foils, sheets, meshes, rods, tubes, single crystals, and porous foams, either supported or unsupported.

The invention will be described further by way of example only with reference to the accompanying drawings, in which:

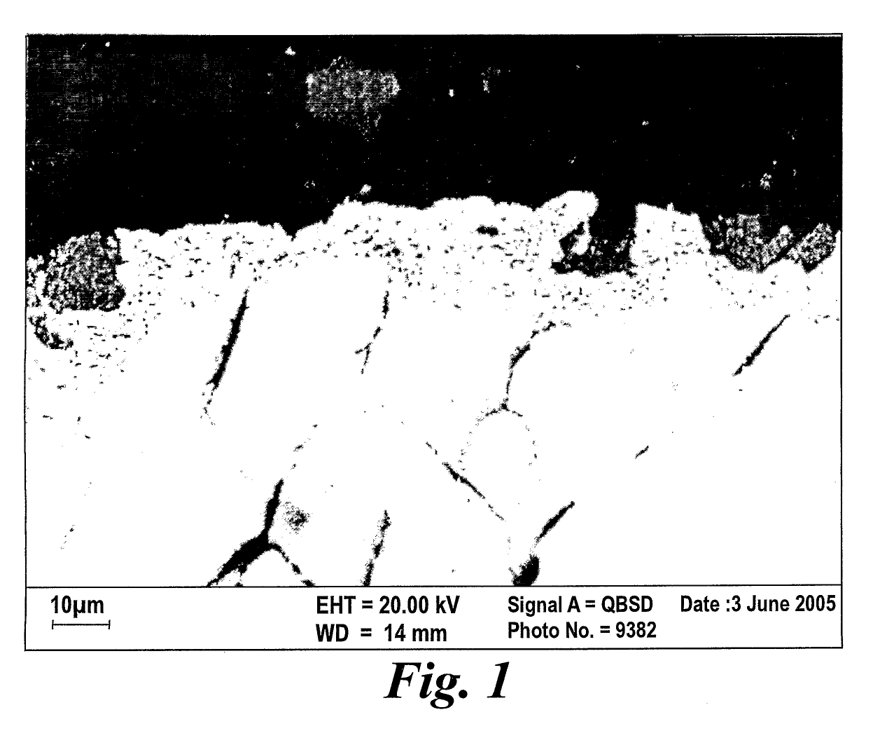

FIG. 1 shows a Field Emission Gradient Scanning Electron Microscopy (FEGSEM) picture of a cross-section of a nickel sphere near the inlet of the gas stream as described in Example 1;

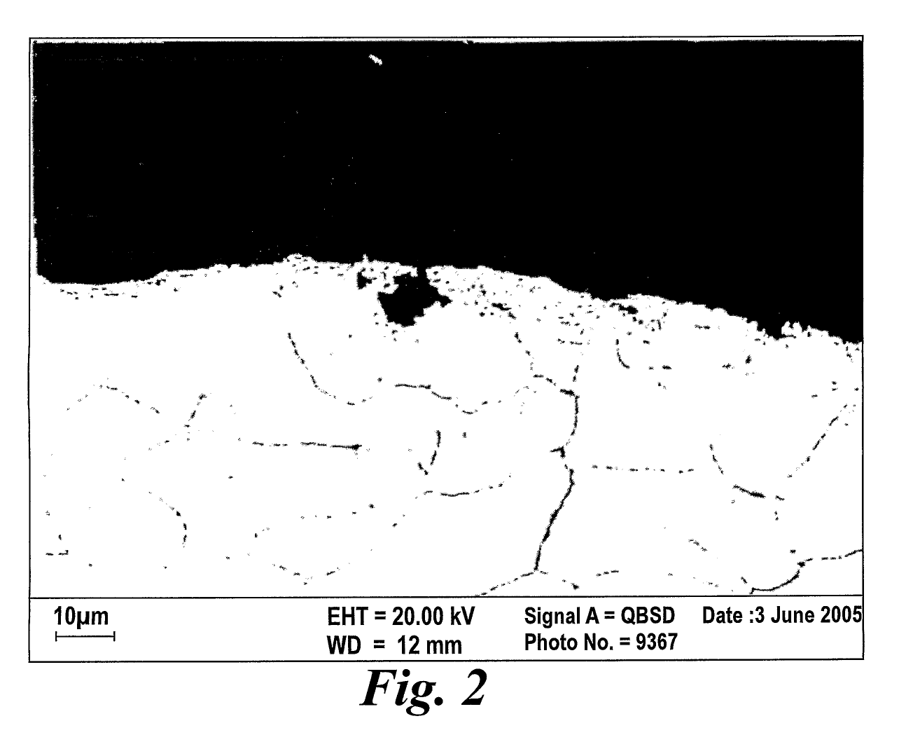

FIG. 2 shows Field Emission Gradient Scanning Electron Microscopy (FEGSEM) picture of a cross-section of a nickel sphere near the outlet of the gas stream as described in Example 1;

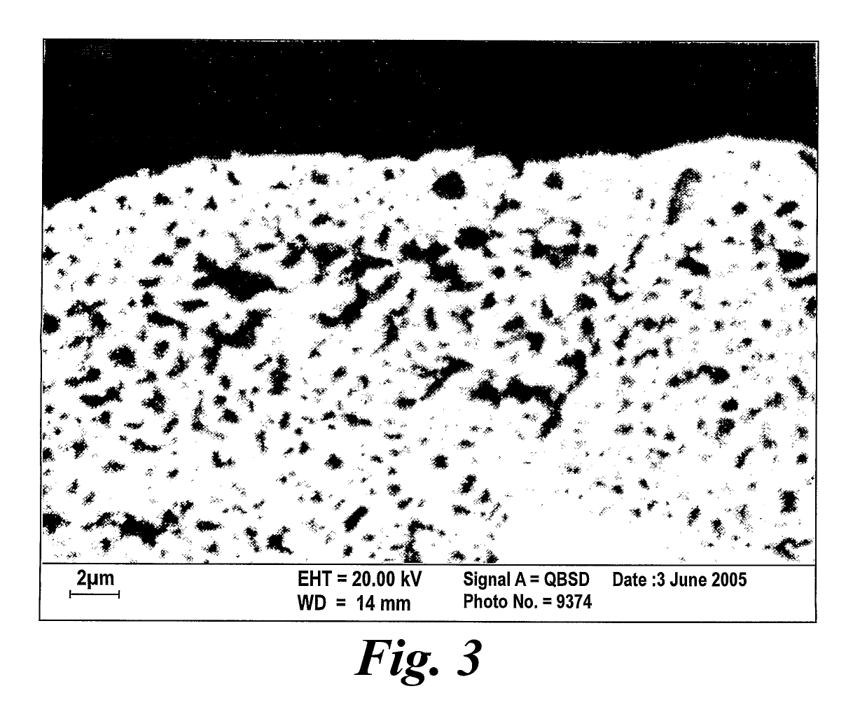

FIG. 3 shows a further Field Emission Gradient Scanning Electron Microscopy (FEGSEM) picture of a cross-section of a nickel sphere near the inlet of the gas stream as described in Example 1; and

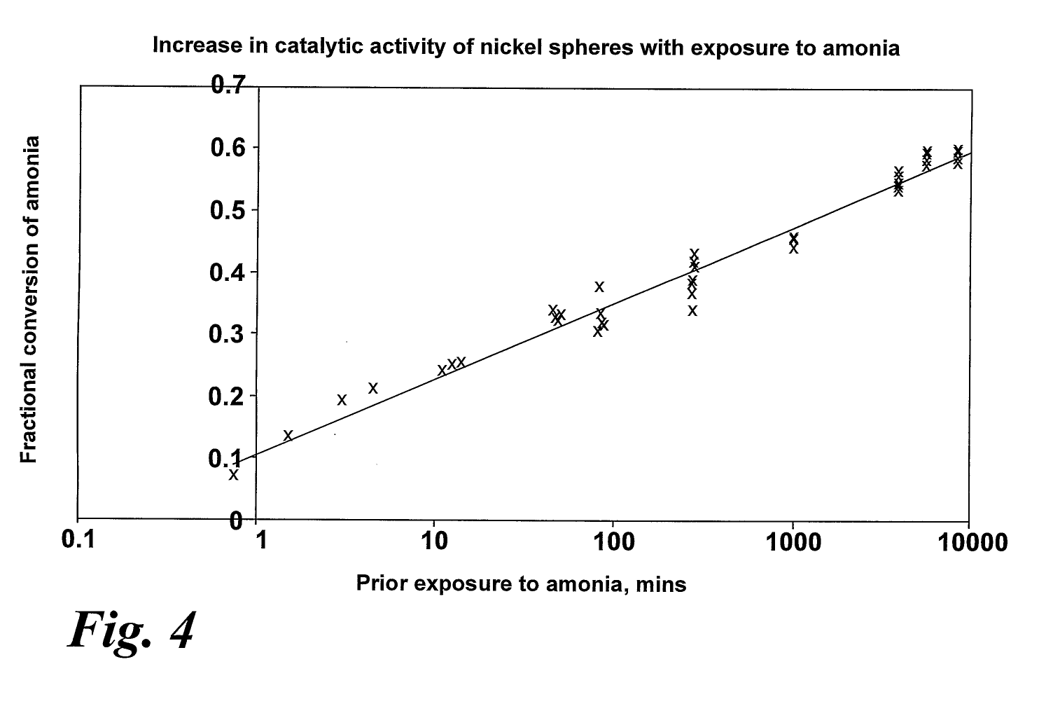

FIG. 4 shows how the catalytic activity of exposed nickel substrates increases as measured by fractional conversion.

### EXAMPLES

### Example 1

Nickel spheres from Goodfellow (99%), [about 0.76 mm diameter] were exposed to pure ammonia (BOC nitride grade) at 600° C. for approximately 140 hours. The spheres were placed in a 4 mm diameter 20 mm long ID quartz tube reactor and held in place with plugs of quartz wool. The tube was placed in a furnace and the spheres were kept under flowing argon until a temperature of 600° C. was reached; the argon was then replaced with pure ammonia flowing at 2 ml/minute.

Before removing the spheres, this procedure was reversed so that the flow was switched over to argon and the tube rapidly cooled to near room temperature. The surface of the spheres was then examined by FEGSEM, and the spheres were sectioned and examined. The results are shown in FIGS. 1 to 3; FIGS. 1 and 3 are cross-sections of a sphere from near the inlet of the reactor tube, with FIG. 3 being a higher magnification micrograph, and FIG. 2 is a cross-section of a sphere from near the outlet of the reactor tube.

On removal from the tube, the spheres were found to be stuck together. They had also lost their shine; on closer inspection those nearer the inlet end of the tube appeared darker than those at the outlet end.

The electron microscope pictures revealed, the development of porosity on the surface of the sphere. Spheres nearer the inlet of the tube had developed a higher degree of porosity, in terms of the depth of porosity and also the degree of porosity developed than those nearer to the outlet end of the tube.

Further scanning electron microscope pictures were taken of cross-sections of spheres which showed development of cracks along the grain boundaries. They also showed the depth of the porous layer is somewhat irregular and several microns deep, and confirm that spheres towards the inlet end of the tube have been more severely attacked.

### Example 2

A similar experiment was conducted to Example 1, except that nitrogen, hydrogen and mixtures of the two were used instead of ammonia.

The resultant nickel surface displayed a much less porous and pitted surface with any pores or pitting thought to be due to the presence of impurities in the nickel, and the formation of resultant nitrides with these. The comparative example demonstrates it is the decomposition of ammonia on the nickel surface which provides the porous structure.

### Example 3

A similar experiment was conducted to Example 1, except that 99.99% pure nickel wire was utilised. The resultant surface had a similar degree of pore formation and pitting to that found for Example 1. This demonstrates that the changes to the spheres observed in Example 1 are not exclusively attributable to the presence of impurities.

1. A method of providing a porous surface on a nickel substrate comprising treating the substrate with a flowing stream of gas comprising ammonia or hydrazine at a temperature of at least 400° C., to provide a porous surface comprising pores substantially all of which have access to the surface.

2. (canceled)

3. (canceled)

4. (canceled)

5. A method of claim 1 wherein the gas stream contains less than 30% water.

6. (canceled)

7. (canceled)

8. A method of claim 5 wherein the nickel substrate has a nickel purity of greater than 98.5%.

9. (canceled)

10. A method of claim 8 wherein the treatment time is 10 to 1000 hours.

11. (canceled)

12. A method of claim 10 wherein the gas stream has a pressure of 1 to 5 bar.

13. (canceled)

14. (canceled)

15. (canceled)

16. A method of claim 12 wherein the gas stream consists essentially of ammonia and is free of oxidising agents and sulphiding agents.

17. (canceled)

18. (canceled)

19. (canceled)

20. A nickel substrate prepared by the method of claim 1.

21. A nickel substrate of claim 20, wherein the substrate has a surface porous layer up to about 30 microns thick.

22. (canceled)

23. A nickel substrate of claim 21 wherein the surface has cracks along the grain boundaries.

24. A nickel substrate of claim 21 wherein the surface of the substrate has pores with a surface pore diameter less than 10 microns.

25. A nickel substrate according to claim 21 wherein the surface of the substrate has pores with a surface pore diameter less than 1 micron.

26. (canceled)

27. (canceled)

28. A nickel substrate prepared by the method of claim 1 in the form of a sphere, powder, wire, foil, sheet, mesh, rod, tube, shape memory article, single crystal or foam, either supported or unsupported.

29. (canceled)

30. (canceled)

31. (canceled)

32. A nickel catalyst comprising a surface prepared by the method of claim 1.

33. (canceled)

34. (canceled)

35. (canceled)

36. A nickel catalyst of claim 32 which is a Raney nickel catalyst.

37. (canceled)

38. A method of steam reforming of hydrocarbons, dry reforming of biogas, hydrogenation of sugars, steam reforming, methanation, activation of fuel in high temperature fuel cell devices or catalytically hydrogenating fatty acids in fats and oils comprising using a catalyst of claim 32.

39. A method of increasing the area of metal-solid-gas interface in fuel cell membranes comprising using a nickel substrate produced by the method of claim 1.

40. A method of increasing the surface area of a nickel powder comprising providing a porous surface in the nickel powder by a method of claim 1.

41. A method of making a porous substrate comprising taking a coarse porous substrate, plating nickel onto the substrate, and providing a porous surface on the nickel by the method of claim 1.

42. (canceled)

43. A method wherein a nickel catalyst or electrode is refreshed continuously during operation by addition of ammonia or hydrazine to the process gas.

44. A method of claim 43 wherein the gas stream comprises less than 0.1% ammonia or hydrazine.
